(19) United States
(12) Patent Application Publication
(10) Pub. No.: US 2004/0002600 A1
(43) Pub. Date: Jan. 1, 2004

(54) Process for the conversion of penam ring system to cepham ring system

(75) Inventors: Pandurang Balwant Deshpande, Chennai (IN); Senthilkumar Udayampalayam Palanisamy, Erode (IN); Padmanabhan Ramar, Thirunelvelli (IN)

Correspondence Address: OLIFF & BERRIDGE, PLC, P.O. BOX 19928, ALEXANDRIA, VA, 22320, US

(73) Assignee: Orchid Chemicals and Pharmaceuticals Limited, Tamilnadu (IN)

(21) Appl. No.: 10/207,110

(22) Filed: Jul. 30, 2002

(30) Foreign Application Priority Data
Jun. 20, 2002 (IN) 467/MAS/2002

(51) Int. Cl. C07D 501/02; C07D 501/10

(52) U.S. Cl. 540/218

(57) ABSTRACT

The present invention relates to a new process for the preparation of cephalosporin derivative of formula (I)

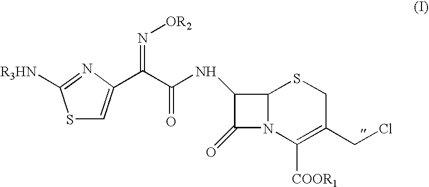

wherein R₁ represents p-methoxybenzyl, p-nitrobenzyl, o-chlorobenzyl or diphenylmethyl; R₂ represents CH₃ or CR^{a}R^{b}COOR^{c} where R^{a} and R^{b} independently represent hydrogen or methyl and R^{c} represents hydrogen or (C₁-C₆) alkyl; R₃ represents hydrogen, acyl, phenacyl, formyl or trityl group.

### FIELD OF INVENTION

The present invention relates to a new process for the preparation of cephalosporin derivative of formula (I)

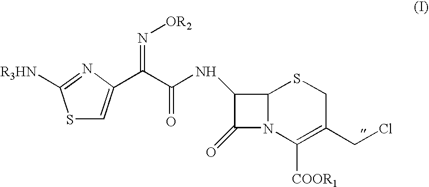

wherein R₁ represents p-methoxybenzyl, p-nitrobenzyl, o-chlorobenzyl or diphenylmethyl; R₂ represents CH₃ or CR^{a}R^{b}COOR^{c} where R^{a} and R^{b} independently represent hydrogen or methyl and R^{c} represents hydrogen or (C₁-C₆)alkyl; R₃ represents hydrogen, acyl, phenacyl, formyl or trityl. The compounds of formula (I) are useful in the preparation of cephalosporin antibiotics of formula (II)

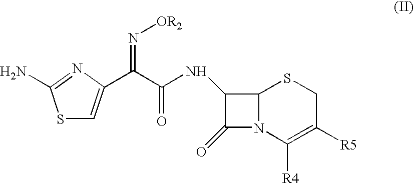

wherein R₄ is carboxylate ion or COOR_{d}, where R_{d} represents hydrogen, ester which form a prodrug or a counter ion which forms a salt; R₂ represents CH₃ or CR^{a}R^{b}COOR^{c} where R^{a} and R^{b} independently represent hydrogen or methyl and R^{c} represents hydrogen or (C₁-C₆)alkyl; R₅ represents CH₃, CH₂OCH₃, CH₂OCOCH₃, CH═CH₂, or

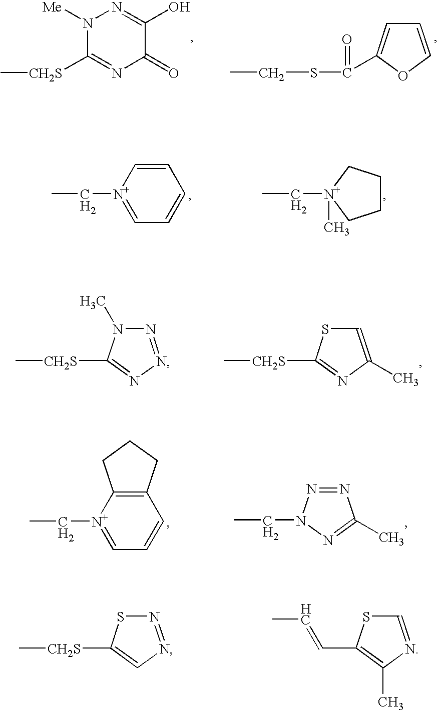

### BACKGROUND OF THE INVENTION

Several patents and publications disclose processes for preparing cephalosporin compounds by condensing the appropriate 7-ACA derivative with respective thiazole group.

U.S. Pat. No. 4,409,214 discloses a process for the preparation of compounds of formula (I) which comprises halogenating the compound of formula (Ia) by conventional halogenation methods.

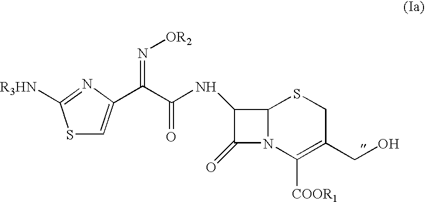

U.S. Pat. No. 4,767,852 discloses a process for the preparation of cephems by acylating 7-amino-3-cephem-4-carboxylic acid with 2-mercaptobenzothiazolyl-(Z)-2-(2-aminothiazol-4-yl)-2-methoxyiminoacetate (MAEM). Similarly, U.S. Pat. No. 5,026,843 (1991) discloses a process for preparing ceftriaxone disodium hemiheptahydrate by acylation of ACT (name) using MAEM as acylating agents in good yield and quality. Thus MAEM has become the standard acylating agent for the preparation of cephalosporins having an oximino group and a 2-aminothiazolyl group in 7-position of cephem compounds.

However, none of the published literature reports a process for the preparation of cephalosporin compounds by the ring expansion concept, wherein a cephem moiety is built up from a penam moiety. We herein report a new methodology for the preparation of Cephalosporin compounds using this concept.

### OBJECTIVES OF THE INVENTION

The primary objective of the invention is to provide a new method for the preparation of Cephalosporin derivatives of the general formula (I), by ring expansion of a penam ring to a cephem ring.

Another objective of the present invention is to provide a process for the preparation of Cephalosporin derivatives of the general formula (I), which would be easy to implement on commercial scales.

Still another objective of the present invention is to provide a process for the preparation of Cephalosporin derivatives of the general formula (I), in good yields with high purity.

Still another objective of the present invention is to provide a process for the preparation of Cephalosporin antibiotics of the general formula (II), using the Cephalosporin derivatives of the general formula (I).

Yet another objective of the present invention is to provide novel intermediates of formula (VI), (VII) (VIII) and (IX), which are useful in the preparation of cephalosporin derivatives.

### SUMMARY OF THE INVENTION

Accordingly, the present invention provides a new process for the preparation of cephalosporin derivatives of formula (I)

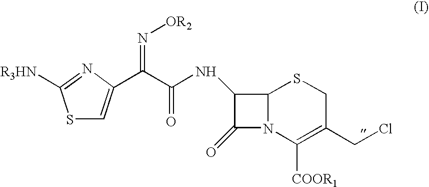

wherein R₁ represents p-methoxybenzyl, p-nitrobenzyl, o-chlorobenzyl or diphenylmethyl; R₂ represents CH₃ or CR^{a}R^{b}COOR^{c} where R^{a} and R^{b} independently represent hydrogen or methyl and R^{c} represents hydrogen or (C₁-C₆)alkyl; R₃ represents hydrogen, acyl, phenacyl, formyl or trityl, which comprises:

(i) condensing the compound of formula (IV) wherein R₂, R₃ are as defined above and R₆ represents hydroxy or an activation group with penam moiety of formula (III) using a base in the presence of a solvent at a temperature in the range of 0° C. to 50° C. to produce a compound of formula (V), wherein R₂ and R₃ are as defined above,

(ii) esterifying the compound of formula (V) using an esterifying agent in the presence of a solvent and a base at a temperature in the range of 25° C. to 50° C. followed by oxidation using an oxidizing agent in the presence of a solvent at a temperature in the range of −16° C. to 10° C. to produce a compound of formula (VI) wherein R₁, R₂ and R₃ are as defined above,

(iii) opening the penam ring of formula (VI) using a mercaptan in the presence of a solvent at a temperature in the range of 80° C. to 120° C. to produce a compound of formula (VII) wherein R₇ represents a heteroaryl ring system and all other symbols are as defined above,

(iv) converting a compound of formula (VII) to a compound of formula (VIII) wherein R₈ represents (C₁-C₆)alkyl or aryl group and all other symbols are as defined above using a metal salt of aryl or alkyl sulfinic acid and a solvent at a temperature in the range of 25° C. to 40° C.,

(v) chlorinating the compound of formula (VIII) using electrochemical methods in a biphasic solvent system at a temperature in the range of 15° C. to 40° C. to produce a compound of formula (IX), where all symbols are as defined above,

(vi) cyclizing the compound of formula (IX) using a base in the presence of a solvent at a temperature in the range of −10° C. to −50° C. to produce a compound of formula (I), where R₁, R₂, and R₃ are as defined above.

The process is shown in Scheme-1

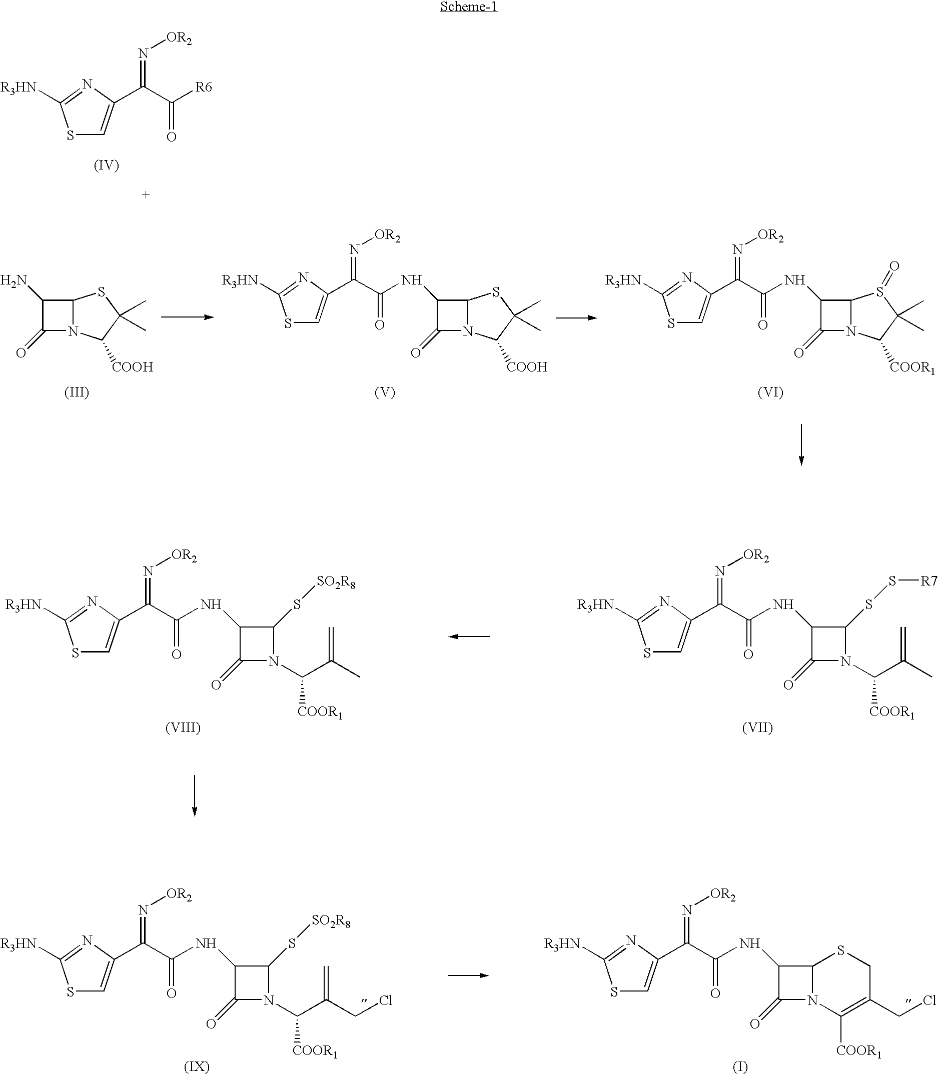

In another embodiment of the present invention there is provided a novel intermediate of formula (VI)

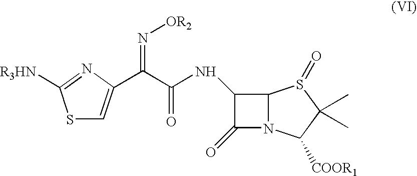

wherein R₁ represents p-methoxybenzyl, p-nitrobenzyl, o-chlorobenzyl or diphenylmethyl; R₂ represents CH₃ or CR^{a}R^{b}COOR^{c} where R^{a} and R^{b} independently represent hydrogen or methyl and R^{c} represents hydrogen or (C₁-C₆)alkyl; R₃ represents hydrogen, acyl, phenacyl, formyl or trityl group.

In another embodiment of the present invention there is provided a novel intermediate of formula (VII)

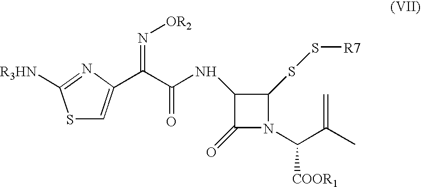

wherein R₁ represents p-methoxybenzyl, p-nitrobenzyl, o-chlorobenzyl or diphenylmethyl; R₂ represents CH₃ or CR^{a}R^{b}COOR^{c} where R^{a} and R^{b} independently represent hydrogen or methyl and R^{c} represents hydrogen or (C₁-C₆))alkyl; R₃ represents hydrogen, acyl, phenacyl, formyl or trityl group; R₇ represents a heteroaryl ring system.

In another embodiment of the present invention there is provided a novel intermediate of formula (VIII)

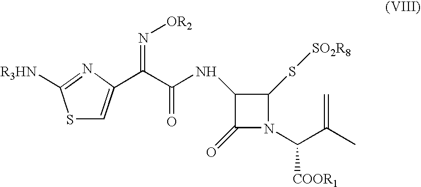

wherein R₁ represents p-methoxybenzyl, p-nitrobenzyl, o-chlorobenzyl or diphenylmethyl; R₂ represents CH₃ or CR^{a}R^{b}COOR^{c} where R^{a} and R^{b} independently represent hydrogen or methyl and R^{c} represents hydrogen or (C₁-C₆)alkyl; R₃ represents hydrogen, acyl, phenacyl, formyl or trityl; R₈ represents (C₁-C₆)alkyl or aryl group.

In yet another embodiment of the present invention there is provided a novel intermediate of formula (IX)

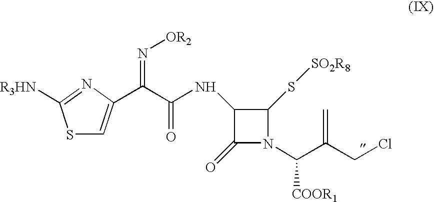

wherein R₁ represents p-methoxybenzyl, p-nitrobenzyl, o-chlorobenzyl or diphenylmethyl; R₂ represents CH₃ or CR^{a}R^{b}COOR^{c} where R^{a} and R^{b} independently represent hydrogen or methyl and R^{c} represents hydrogen or (C₁-C₆)alkyl; R₃ represents hydrogen, acyl, phenacyl, formyl or trityl group; R₈ represents (C₁-C₆)alkyl or aryl group.

In still another embodiment of the present invention there is provided a process for the preparation of cephalosporin antibiotics of formula (II)

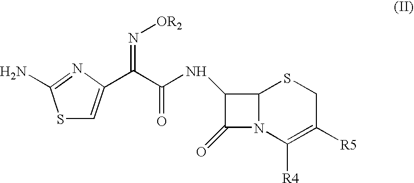

wherein R₄ is carboxylate ion or COOR_{d}, where R_{d} represents hydrogen, ester which form a prodrug or a counter ion which forms a salt; R₂ represents CH₃ or CR^{a}R^{b}COOR^{c} where R^{a} and R^{b} independently represent hydrogen or methyl and R^{c} represents hydrogen or (C₁-C₆)alkyl; R₅ represents CH₃, CH₂OCH₃, CH₂OCOCH₃, CH═CH₂, or

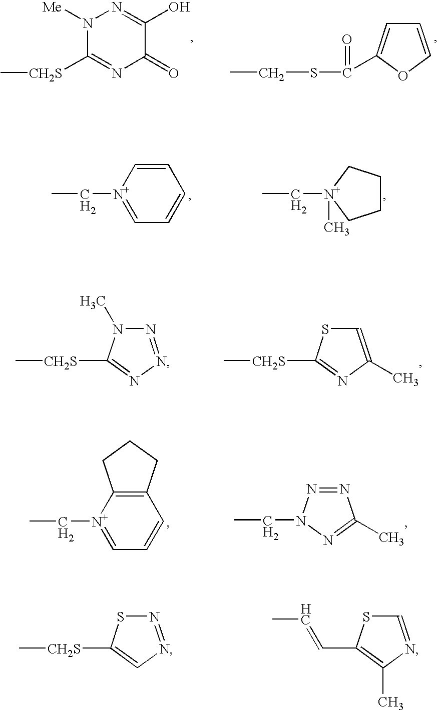

from a compound of formula (I).

### DETAILED DESCRIPTION OF THE INVENTION

In an embodiment of the present invention the heteroaryl group represented by R₇ is selected from 2-mercaptobenzothiazole, 2-mercaptobenzooxazole, 2-mercaptobenzimidazole or 2-mercapto-5-methyltetrazole.

In still another embodiment of the present invention the counter ion represented by R_{d} is alkali metal, preferably sodium.

In still another embodiment of the present invention the prodrug ester represented by R_{d} is —(CH₂)—O—C(═O)—C(CH₃)₃, —CH(CH₃)—O—C(═O)—CH₃ or —CH(CH₃)—O—C(═O)—O—CH(CH₃)₂.

In still another embodiment of the present invention the groups represented by R₈ are selected from (C₁-C₆)alkyl group such as methyl, ethyl, n-propyl, iso-propyl, butyl, iso-butyl, sec-butyl; aryl group such as phenyl, p-methylphenyl.

In another embodiment of the present invention the compound of formula (I) obtained is a syn-isomer.

In another embodiment of the present invention the condensation in step (i) is carried out using water and any water miscible solvent selected from tetrahydrofuran, acetone, acetonitrile, dioxane, DMF, DMAc and alcohols such as methanol, ethanol, iso-propanol, in the presence of a base such as sodium acetate, triethylamine, diethylamine at a temperature in the range of 0 to 50° C.

The activation group used in the compound of formula (IV) is selected from esters, thioesters, anhydrides or halides; which are reported in the literature.

In yet another embodiment of the present invention the esterification in step (ii) is carried out using esterifying agents such as p-methoxybenzyl bromide, p-methoxybenzyl chloride, p-nitrobenzyl bromide, p-nitrobenzyl chloride, o-chlorobenzyl chloride in the presence of a base selected from alkali and alkaline earth metal carbonates and hydroxides such as sodium carbonate, potassium carbonate, sodium hydroxide, potassium hydroxide and the like, and a solvent selected from methylenedichloride, dimethyl formamide, acetonitrile, dioxane, tetrahydrofuran, ethyl acetate or dimethyl acetamide.

The esterification in step (ii) is also carried out using esterifying agents such as diphenyl diazomethane generated from benzophenone hydrazone in an acid medium and a solvent selected from methylene dichlonide, chloroform, ethyl acetate, toluene, water, in the presence of catalytic quantities of iodine.

The oxidation to obtain compounds of formula (VI) is carried out using peracetic acid, m-chloroperbenzoic acid, H₂O₂, trifluoroperacetic acid, magnesium monoperoxy phthalate and the solvent is selected from methylenedichloride, chloroform, toluene, dimethyl formamide, ethyl acetate, acetic acid, dimethyl acetamide, acetone or dioxane.

The compound of formula (VI) wherein R₃ represents hydrogen may be converted to compounds of formula (VI) wherein R₃ represents acyl, phenacyl, formyl, trityl before further progressing with the reaction. The conversion is carried out using acetic anhydride, formic acetic anhydride, acid chloride, trityl chloride in the presence of a solvent selected from THF, methylenedichloride, dioxane, acetonitrile, THF, toluene or acetic acid.

In yet another embodiment of the present invention the ring opening in step (iii) is carried out using a mercaptan selected from 2-mercaptobenzothiazole, 2-mercaptobenzooxazole, 2-mercaptobenzimidazole, 2-mercapto-5-methyltetrazole and the like, in the presence of a solvent selected from 1,4-dioxane, toluene or xylene.

In yet another embodiment of the present invention the conversion in step (iv) is carried out using metal salt of aryl or alkyl sulfinic acid selected from Copper (II) p-toluenesulfinate, Copper (II) benzenesulfinate, Silver (II) p-toluenesulfinate, Silver (II) benzenesulfinate, Copper (II) methanesulfinate, Silver (II) methanesulfinate, and the like in the presence of a solvent selected from acetone, THF, dioxane, acetonitrile, alcohols such as methanol, ethanol or iso-propanol, with or without water.

In yet another embodiment of the present invention the electrochemnical chlorination in step (v) is carried out using sodium chloride containing catalytic amounts of conc. sulphuric acid. The reaction is carried in a biphasic solvent system selected from chloroform, methylene dichloride, carbon tetrachloride, with or without ethyl acetate as a co-solvent.

In yet another embodiment of the present invention the cyclisation in step (vi) is carried out using a base selected from ammonia, ammonium salts like ammonium carbonate, ammonium acetate, organic amines like di-isopropylamine, diethylamnine, methylamine, triethylamine and the like in the presence of a solvent selected from DMF, acetonitrile, dimethyl acetamide, ethyl acetate, dioxane, THF or methylene dichloride.

Many other beneficial results can be obtained by applying disclosed invention in a different manner or by modifying the invention with the scope of disclosure.

The present invention is provided by the examples below, which are provided by way of illustration only and should not be considered to limit the scope of the invention.

### EXAMPLE 1

### Preparation of 6-(2-(2-Aminothiazol-4-yl)-2-(syn-methoxyimino) Acetamido) Penicillanic Acid

To cold aq. THF (1000 ml), 6-aminopenicillanic acid (50 gm) was added followed by S-benzothiazol-2-yl 2-(2-aminothiazol-4-yl)-2-(syn-methoxyimino)thioacetate (88.4 gm). To the reaction mixture, a solution of triethylamine (24.6 gm) in THF was added over a period of 60 minutes. The reaction mixture was maintained for 4-6 hours. After the reaction was complete, ethyl acetate was added to the reaction mixture. The product was extracted in to aqueous layer and treated with charcoal. The filtrate was acidified with dil. HCl, filtered and washed with 2-propanol (600 ml). The product was dried to get pure 6-(2-(2-aminothiazol-4-yl)-2-(syn-methoxyimino)acetamido)penicillanic acid.

¹H NMR (DMSO-d₆, δ ppm): 1.48 (3H, s), 1.59 (3H, s), 3.82 (3H, s), 4.23 (3H, s), 5.52 (1H, d), 5.58 (1H, dd), 6.76 (1H, s), 7.21 (2H, s), 9.55 (1H, d), & 13.0 (1H, bs). Mass (M/e): M+1: 400.2.

### EXAMPLE 2

### Preparation of p-Methoxybenzyl 6-(2-(2-aminothiazol-4-yl)-2-(syn-methoxyimino) Acetamido) Pencillanate-1-oxide

To N,N-dimethylacetamide (125 ml), 6-(2-(2-aminothiazol-4-yl)-2-(syn-methoxyimino)acetamido)penicillanic acid (26.5 gm) and sodium carbonate (5.1 gm) were added at 27° C. under dry condition. The reaction mixture was stirred for 15-20 minutes. Potassium bromide (8.5 gm), and p-methoxybenzyl chloride (11.0 gm) were added at 25-27° C. and maintained until the reaction was over. The reaction mixture was poured into a mixture of cold water and isopropyl ether, and filtered. The product was taken in cold dichloromethane (250 ml) and oxidized with peracetic acid (21 ml). After the reaction was complete, the organic layer was separated and washed with sodium thiosulphate solution, water, and saturated sodium bicarbonate solution. The dichloromethane layer was treated with charcoal, concentrated under vacuum and stirred with dichloroethane and isopropyl ether (150 ml). The product obtained was filtered and dried under vacuum to get p-methoxybenzyl 6-(2-(2-aminothiazol-4-yl)-2-(syn-methoxyimino)acetamido)penicillanate-1-oxide. ¹H NMR (CDCl₃, δ ppm): 1.05 (3H, s), 1.63 (3H, s), 3.81 (3H, s), 4.03 (3H, s), 4.65 (1H, s), 5.08 (1H, d), 5.09 (1H, d), 5.25 (1H, d), 5.7 (1H, bs), 6.11 (1H, dd), 6.88 (2H, d), 6.92 (1H, s), 7.32 (2H, d), & 7.83 (1H, d). Mass (M/e): M+1: 536.1

### EXAMPLE 3

### Preparation of p-Methoxybenzyl 6-(2-(2-formylaminothiazol-4-yl)-2-(syn-methoxyimino) Acetamido) Pencillanate-1-oxide

p-Methoxybenzyl 6-(2-(2-aminothiazol-4-yl)-2-(syn-methoxyimino) acetamido) pencillanate-1-oxide (25 gm) and THF were added to formic acetic anhydride, and stirred at RT for 1-2 hours. After the reaction was complete, the reaction mixture was poured into isopropyl ether (500 ml) and stirred for 15-30 minutes. The reaction mixture was filtered and washed with isopropyl ether (100 ml), water and sodium bicarbonate solution (250 ml). The material was dissolved in dichloromethane (250 ml), and treated with charcoal. The filtrate was concentrated under vacuum, treated with isopropyl ether, filtered and dried under vacuum to afford p-methoxybenzyl 6-(2-(2-formylaminothiazol-4-yl)-2-(syn-methoxyimino)acetamido)pencillanate-1-oxide. ¹H NMR (CDCl₃, δ ppm): 1.11 (3H, s), 1.13 (3H, s), 3.81 (3H, s), 4.01 (3H, s), 4.66 (1H, s), 5.11 (1H, d), 5.12 (1H, d), 5.25 (1H, d), 6.13 (1H, dd), 6.89 (2H, d), 7.31 (2H, d), 7.40 (1H, s), 8.05 (1H, d), 8.64 (1H, s) & 12.0 (1H, bs). Mass (M/e): M+1: 564.2

### EXAMPLE 4

### Preparation of p-Methoxybenzyl 2-(2-Benzothiazolyldithio)-α-(1-methylethenyl)-4-oxo-3-(2-(2-formylaminothiazol-4-yl)-2-(syn-methoxyimino)acetamido)-1-azetidineacetate

To 1,4-dioxane (300 ml) contained in a RB flask, p-methoxybenzyl 6-(2-(2-formylaminothiazol-4-yl)-2-(syn-methoxyimino)acetamido)pencillanate-1-oxide (15 gm) and 2-mercaptobenzothiazole (4.5 gm) were added at 27° C. under nitrogen. The reaction mixture was heated under reflux over a period of 30 minutes and maintained at reflux temperature over 5 hours with slow distillation of the solvent (The reaction may also be carried out in toluene as a solvent with a conventional Dean-Stark set up, with continuous removal of water). After the reaction was over, the solvent was removed under vacuum, treated with isopropyl ether, filtered and dried under vacuum to afford p-methoxybenzyl 2-(2-benzothiazolyldithio)-α-(1-methylethenyl)-4-oxo-3-(2-(2-formylaminothiazol-4-yl)-2-(syn-methoxyimino)acetamido)-1-azetidineacetate, which was taken to next step.

### EXAMPLE 5

### Preparation of p-Methoxybenzyl 2-(2-Tolueneysulfonylthio)-α-(1-methylethenyl)-4-oxo-3-(2-(2-formylaminothiazol-4-yl)-2-(syn-methoxyimino)acetamido)-1-azetidineacetate

p-Methoxybenzyl 2-(2-benzothiazolyldithio)-α-(1-methylethenyl)-4-oxo-3-(2-(2-formylaminothiazol-4-yl)-2-(syn-methoxyimino)acetamido)-1-azetidineacetate (5.5 gm) was taken in aqueous acetone (100 mL) at 27-30° C. Copper (II) p-toluene sulfinate (2.24 gm) was added to the mixture. The contents of the reaction mixture were heated under reflux and maintained for 30 minutes. After the reaction was over, the reaction mixture was filtered, concentrated and taken in ethyl acetate. The organic layer was washed with water, treated with charcoal and worked up in the usual manner to yield p-methoxybenzyl 2-(2-toluenesulfonylthio)-α-(1-methylethenyl)-4-oxo-3-(2-(2-formylaminothiazol-4-yl)-2-(syn-methoxyimino)acetamido)-1-azetidineacetate. ¹H NMR (CDCl₃, δ ppm): 1.76 (3H, s), 2.43 (3H, s), 3.78 (3H, s), 3.81 (3H, s), 4.48 (1H, s), 4.77 (1H, s) & 4.83 (1H, s), 5.09 (2H, ABq), 5.50 (1H, dd), 5.94 (1H, d), 6.88 (2H, d), 7.26 (1H, s), 7.27 (2H, d), 7.30 (2H, d), 7.75 (2H, s), 8.18 (1H, bs), 8.53 (1H, bs) & 11.0 (1H, bs). Mass (M/e): M+1: 702.3

### EXAMPLE 6

### Preparation of p-Methoxybenzyl 2-(2-Toluenesulfonylthio)-α-(1-chloromethylethenyl)-4-oxo-3-(2-(2-formylaminothiazol-4-yl)-2-(syn-methoxyimino)acetamido)-1-azetidineacetate

p-Methoxybenzyl 2-(2-toluenesulfonylthio)-α-(1-methylethenyl)-4-oxo-3-(2-(2-formylaminothiazol-4-yl)-2-(syn-methoxyimino)acetamido)-1-azetidineacetate (5.0 gm) was added to a mixture of chloroform and ethyl acetate at 26-28° C. An aqueous solution of sodium chloride containing catalytic quantities of conc. sulphuric acid was added. The biphasic reaction mixture was placed in an electrochemical unit equipped with an undivided cell. Electrolysis was carried out using precious metal oxide coated over expanded mesh, as anode. Appropriate electric charge of 6F to 11F was passed while maintaining effective stirring. After the reaction was over, organic layer was separated, and washed with a solution of sodium thiosulphate followed by water. The organic layer was treated with charcoal, concentrated and worked up as usual to get p-methoxybenzyl 2-(2-toluenesulfonylthio)-α-(1-chloromethylethenyl)-4-oxo-3-(2-(2-formylaminothiazol-4-yl)-2-(syn-methoxyimino)acetamido)-1-azetidineacetate, which was taken to next step without purification.

### EXAMPLE 7

### Preparation of p-Methoxybenzyl 7-(2-(2-Formylaminothiazol-4-yl)-2-(syn-methoxyimino)acetamido)-3-chloromethyl-3-cephem-4-carboxylate

p-Methoxybenzyl 2-(2-toluenesulfonylthio)-α-(1-chloromethylethenyl)-4-oxo-3-(2-(2-formylaminothiazol-4-yl)-2-(syn-methoxyimino)acetamido)-1-azetidineacetate (3.3 gm) was added to DMF (16.5 mL) and cooled to −35° C. A solution of ammonia (1 ml) in DMF was added and maintained until the reaction was completed. The reaction mixture was acidified with dil. HCl, filtered. The solid obtained was extracted with ethyl acetate, treated with charcoal, concentrated and treated with methanol to get p-methoxybenzyl 7-(2-(2-formyl aminothiazol-4-yl)-2-(syn-methoxyimino)acetamido)-3-chloromethyl-3-cephem-4-carboxylate.

### EXAMPLE 8

### Preparation of p-Methoxybenzyl 7-(2-(2-Formylaminothiazol-4-yl)-2-(syn-methoxyimino)acetamido)-3-(5-methyl-1,3,4-thiadiazolyl-2-thiomethyl)-3-cephem-4-carboxylate

2-Mercapto-5-methyl-1,3,4-thiadiazole (6.27 gm) was dissolved in sodium hydroxide solution (1.81 gm in 15.0 ml water) at 28-30° C. and stirred at this temperature for 30 min. The clear solution was added to a cold solution of p-methoxybenzyl 7-(2-(2-formylaminothiazol-4-yl)-2-(syn-methoxyimino)acetamido)-3-chloromethyl-3-cephem-4-carboxylate (25.0 gm) in DMF (125 ml). The progress of the reaction was monitored. After the reaction was over, the reaction mixture was poured into cold water and the product obtained was isolated by conventional methods to get 28-29 gm of pure p-methoxybenzyl 7-(2-(2-formylaminothiazol-4-yl)-2-(syn-methoxyimino)acetamido)-3-(5-methyl-1,3,4-thiadiazolyl-2-thiomethyl)-3-cephem-4-carboxylate.

### EXAMPLE 9

### Preparation of p-Methoxybenzyl 7-(2-(2-Formylaminothiazol-4-yl)-2-(syn-methoxyimino)acetamido)-3-(1-pyridiniomethyl)-3-cephem-4-carboxylate

p-Methoxybenzyl 7-(2-(2-formylaminothiazol-4-yl)-2-(syn-methoxyimino)acetamido)-3-chloromethyl-3-cephem-4-carboxylat (25.0 gm) was dissolved in acetone (150 ml) at 28-30° C. under dry condition. To the clear solution, sodium iodide (6.79 gm) was added and stirred well. Pyridine (3.58 gm) was added and stirred while monitoring the progress of the reaction. After the reaction was over, the reaction mixture was poured into cold water and the product was isolated by conventional methods to get 23-25 gm of pure p-methoxybenzyl 7-(2-(2-formylaminothiazol-4-yl)-2-(syn-methoxyimino)acetamido)-3-(1-pyridiniomethyl)-3-cephem-4-carboxylate.

### EXAMPLE 10

### Preparation of p-methoxybenzyl 7-(2-(2-formylaminothiazol-4-yl)-2-(syn-methoxyimino)acetamido)-3-vinyl-3-cephem-4-carboxylate

p-Methoxybenzyl 7-(2-(2-formylaminothiazol-4-yl)-2-(syn-methoxyimino)acetamido)-3-chloromethyl-3-cephem-4-carboxylate (25.0 gm) was dissolved in N,N-dimethylfoimamide (250 ml) at 28-30° C. under dry condition. To the clear solution, sodium iodide (7.89 gm) and triphenylphosphine (11.77 gm) were added and stirred well. DM water (250 ml), formaldehyde solution (36 ml) and sodium carbonate solution (2.52 gm in 40 ml water) were added and stirred well. The progress of the reaction was monitored. After the reaction was over, the reaction mixture was quenched with cold water and the product isolated by conventional methods to get 20-21 gm of pure p-methoxybenzyl 7-(2-(2-formylaminothiazol-4-yl)-2-(syn-methoxyimino)acetamido)-3-vinyl-3-cephem-4-carboxylate.

1. A new process for the preparation of cephalosporin derivatives of formula (I) 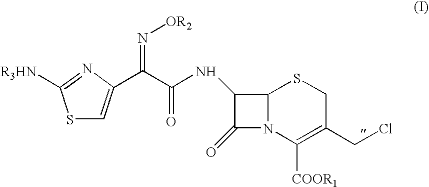
wherein R₁ represents p-methoxybenzyl, p-nitrobenzyl, o-chlorobenzyl or diphenylmethyl; R₂ represents CH₃ or CR^{a}R^{b}COOR^{c} where R^{a} and R^{b} independently represent hydrogen or methyl and R^{c} represents hydrogen or (C₁-C₆)alkyl; R₃ represents hydrogen, acyl, phenacyl, formyl or trityl, the said process comprising steps of:
(i) condensing the compound of formula (IV) 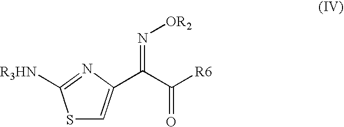
wherein R₂, R₃ are as defined above and R₆ represents hydroxy or an activation group with penam moiety of formula (III) 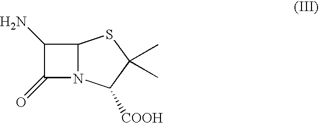
using a base in presence of a solvent at a temperature in the range of 0° C. to 50° C. to produce a compound of formula (V), 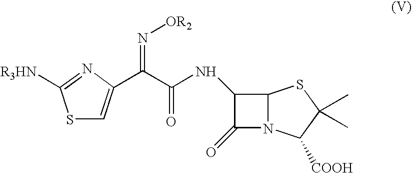
wherein R₂ and R₃ are as defined above,
(ii) esterifying the compound of formula (V) using an esterifying agent in the presence of a solvent and a base at a temperature in the range of 25° C. to 50° C. followed by oxidation using an axidising agent in the presence of a solvent at a temperature in the range of −10° C. to 10° C. to produce a compound of formula (VI) 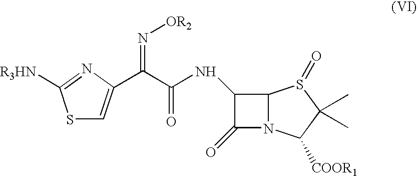
wherein R₁, R₂ and R₃ are as defined above,
(iii) opening the penam ring of formula (VI) using a mercaptan in the presence of a solvent at a temperature in the range of 80° C. to 120° C. to produce a compound of formula (VII) 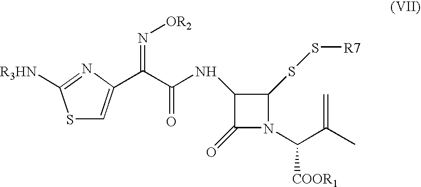
wherein R₇ represents a heteroaryl ring system and all other symbols are as defined above,
(iv) converting a compound of formula (VII) to a compound of formula (VIII) 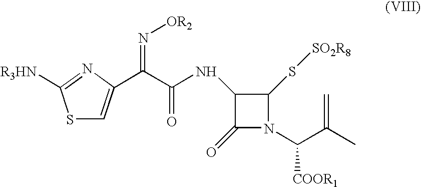
using a metal salt of aryl or alkyl sulfinic acid and a solvent at a temperature in the range of 25° C. to 40° C., wherein R₈ represents (C₁-C₆)alkyl or aryl group and all other symbols are as defined above
(v) chlorinating the compound of formula (VIII) using electrochemical method in a biphasic solvent system at a temperature in the range of 15° C. to 40° C. to produce a compound of formula (IX), 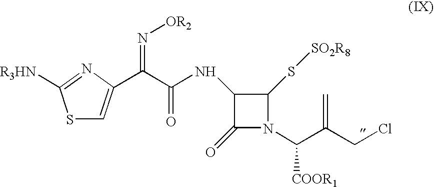
wherein R₈ represents (C₁-C₆)alkyl or aryl group and all other symbols are as defined above,
(vi) cyclizing the compound of formula (IX) using a base in the presence of a solvent at a temperature in the range of −10° C. to −50° C. to produce a compound of formula (I) where R₁, R₂, and R₃ are as defined above.

2. The process of claim 1, the heteroaryl group represented by R₇ is selected from 2-mercaptobenzothiazole, 2-mercaptobenzooxazole, 2-mercaptobenzimidazole or 2-mercapto-5-methyltetrazole.

3. The process of claim 1, the groups represented by R₈ are selected from (C₁-C₆)alkyl group such as methyl, ethyl, n-propyl, iso-propyl, butyl, iso-butyl, sec-butyl; aryl group such as phenyl, p-methylphenyl.

4. The process of claim 1, wherein the solvent used in step (i) is selected from tetrahydrofuran, acetone, acetonitrile, dioxane, DMF, DMAc or alcohol.

5. The process of claim 4, wherein the alcohol is selected from methanol, ethanol or propanol.

6. The process of claim 1, wherein the base used in step (i) is selected from sodium acetate, triethylamine or diethylamine.

7. The process of claim 1, wherein the activation group used in the compound of formula (IV) is selected from esters, thioesters, anhydrides or halides.

8. The process of claim 1, wherein the esterifying agent used in step (ii) is selected from p-methoxybenzyl bromide, p-methoxybenzyl chloride, p-nitrobenzyl bromide, p-nitrobenzyl chloride, o-chlorobenzyl chloride or diphenyl diazomethane.

9. The process of claim 1, wherein the solvent used in step (ii) is selected from methylenedichloride, dimethyl formamide, acetonitrile, dioxane, tetrahydrofuran, ethyl acetate or dimethyl acetamide.

10. The process of claim 1, wherein the base used in step (ii) is selected from alkali and alkaline earth metal carbonates and hydroxides such as sodium carbonate, potassium carbonate, sodium hydroxide or potassium hydroxide.

11. The process of claim 1, wherein the oxidation in step (ii) is carried out using peracetic acid, m-chloroperbenzoic acid, H₂O₂, trifluoroperacetic acid or magnesium monoperoxy phthalate.

12. The process of claim 1, wherein the solvent used in step (ii) for oxidation is selected from methylenedichloride, chloroform, toluene, dimethyl formamide, ethyl acetate, acetic acid, dimethyl acetamide, acetone or dioxane.

13. The process of claim 1, wherein the mercaptan used in step (iii) is selected from 2-mercaptobenzothiazole, 2-mercaptobenzooxazole, 2-mercaptobenzimidazole or 2-mercapto-5-methyltetrazole.

14. The process of claim 1, wherein the solvent used in step (iii) is selected from 1,4-dioxane, toluene or xylene.

15. The process of claim 1, wherein the metal salt used in step (iv) is selected from Copper (II) p-toluenesulfinate, Copper (II) benzenesulfinate, Silver (II) p-toluenesulfinate, Silver (II) benzenesulfinate, Copper (II) methanesulfinate or Silver (II) methanesulfinate.

16. The process of claim 1, wherein the solvent used in step (iv) is selected from acetone, THF, dioxane, acetonitrile and alcohol, with or without water.

17. The process of claim 16, wherein the alcohol is selected from methanol, ethanol or propanol.

18. The process of claim 1, wherein the electrochemical chlorination in step (v) is carried out using sodium chloride containing catalytic amounts of concentrated sulphuric acid.

19. The process of claim 1, wherein the electrochemical chlorination in step (v) is carried out in a biphasic solvent system selected from chloroform, methylene dichloride, carbon tetrachloride, with or without ethyl acetate as a co-solvent.

20. The process of claim 1, wherein the base used in step (vi) is selected from ammonia, ammonium salt or organic amine.

21. The process of claim 20, wherein the ammonium salt is selected from ammonium carbonate or ammonium acetate.

22. The process of claim 20, wherein the organic amine is selected from di-isopropylamine, diethylamine, methylamine or triethylamine.

23. The process of claim 1, wherein the solvent used in step (vi) is selected from DMF, acetonitrile, dimethyl acetamide, ethyl acetate, dioxane, THF or methylene dichloride.

24. A process for the conversion of the compound of formula (VI) 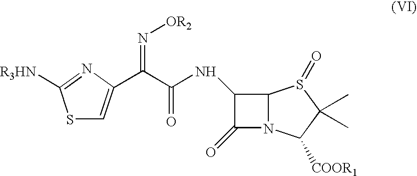
wherein R₃ represents hydrogen; R₁ represents p-methoxybenzyl, p-nitrobenzyl, o-chlorobenzyl or diphenylmethyl; R₂ represents CH₃ or CR^{a}R^{b}COOR^{c} where R^{a} and R^{b} independently represent hydrogen or methyl and R^{c} represents hydrogen or (C₁-C₆)alkyl to a compound of formula (VI) where R₃ represents acyl, phenacyl, formyl or trityl and all other symbols are as defined above using acetic anhydride, formic acetic anhydride, acid chloride or trityl chloride in the presence of a solvent.

25. The process of claim 24, wherein the solvent used is selected from THF, methylenedichloride, dioxane, acetonitrile, THF, toluene or acetic acid.

26. An intermediate of formula (VI) 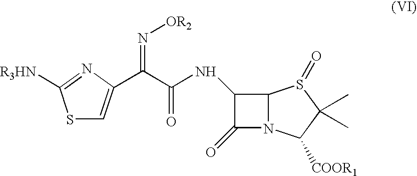
wherein R₁ represents p-methoxybenzyl, p-nitrobenzyl, o-chlorobenzyl or diphenylmethyl; R₂ represents CH₃ or CR^{a}R^{b}COOR^{c} where R^{a} and R^{b} independently represent hydrogen or methyl and R^{c} represents hydrogen or (C₁-C₆)alkyl; R₃ represents hydrogen, acyl, phenacyl, formyl or trityl group.

27. An intermediate of formula (VII) 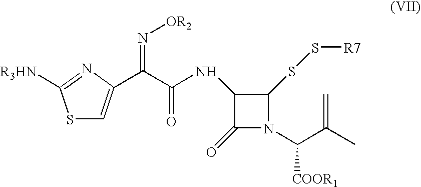
wherein R₁ represents p-methoxybenzyl, p-nitrobenzyl, o-chlorobenzyl or diphenylmethyl; R₂ represents CH₃ or CR^{a}R^{b}COOR^{c} where R^{a} and R^{b} independently represent hydrogen or methyl and R^{c} represents hydrogen or (C₁-C₆)alkyl; R₃ represents hydrogen, acyl, phenacyl, formyl or trityl group; R₇ represents a heteroaryl ring system.

28. An intermediate of formula (VIII) 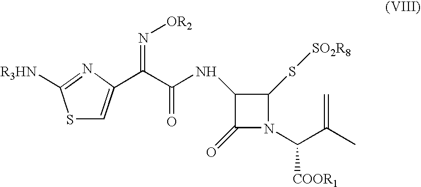
wherein R₁ represents p-methoxybenzyl, p-nitrobenzyl, o-chlorobenzyl or diphenylmethyl; R₂ represents CH₃ or CR^{a}R^{b}COOR^{c} where, R^{a} and R^{b} independently represent hydrogen or methyl and R^{c} represents hydrogen or (C₁-C₆)alkyl; R₃ represents hydrogen, acyl, phenacyl, formyl or trityl; R₈ represents (C₁-C₆)alkyl or aryl group.

29. An intermediate of formula (IX) 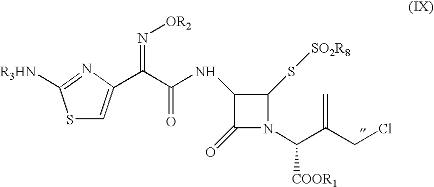
wherein R₁ represents p-methoxybenzyl, p-nitrobenzyl, o-chlorobenzyl or diphenylmethyl; R₂ represents CH₃ or CR^{a}R^{b}COOR^{c} where R^{a} and R^{b} independently represent hydrogen or methyl and R^{c} represents hydrogen or (C₁-C₆)alkyl; R₃ represents hydrogen, acyl, phenacyl, formyl or trityl group; R₈ represents (C₁-C₆)alkyl or aryl group.

30. A process for the preparation of cephalosporin antibiotics of formula (II) 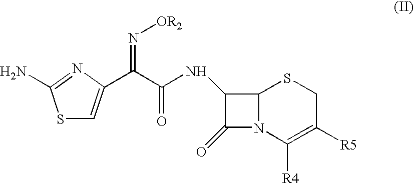
wherein R₄ is carboxylate ion or COOR_{d}, where R_{d} represents hydrogen or ester which form a prodrug or a counter ion, resulting a salt; R₂ represents CH₃ or CR^{a}R^{b}COOR^{c} where R^{a} and R^{b} independently represent hydrogen or methyl and R^{c} represents hydrogen or (C₁-C₆)alkyl; R₅ represents CH₃, CH₂OCH₃, CH₂OCOCH₃, CH═CH₂, or 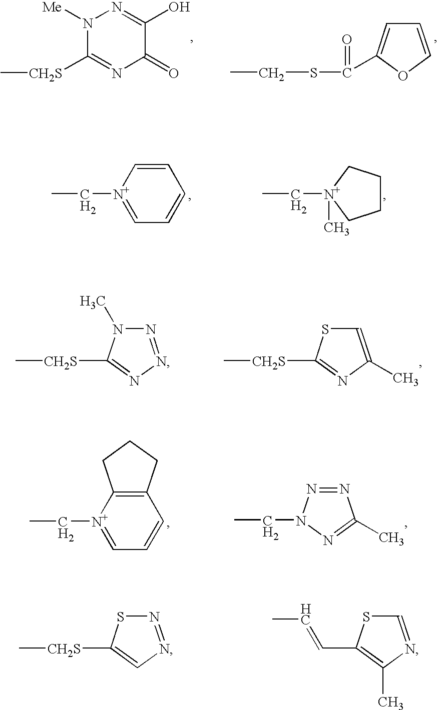
from a compound of formula (I) 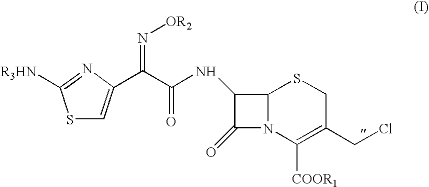
prepared by a process as claimed in any of the preceding claims.

31. The process of claim 30, the counter ion represented by R_{d} is an alkali metal, preferably sodium.

32. The process of claim 30, the prodrug ester represented by R_{d} is —(CH₂)—O—C(═O)—C(CH₃)₃, —CH(CH₃)—O—C(═O)—CH₃ or —CH(CH₃)—O—C(═O)—O—CH(CH₃)₂.
